(19) United States
(12) Patent Application Publication
(10) Pub. No.: US 2013/0303565 A1
(43) Pub. Date: Nov. 14, 2013

(54) USE OF A NEW HISTAMINE H4 AGONIST FOR THE TREATMENT OF ACUTE LEUKEMIA

(71) Applicants: Gaetan TERRASSE, SAINT-VALLIER (FR); Bruno DIOT, MERCUREY (FR); Patrice BINAY, COUCHEY (FR)

(72) Inventors: Gaetan TERRASSE, SAINT-VALLIER (FR); Bruno DIOT, MERCUREY (FR); Patrice BINAY, COUCHEY (FR)

(21) Appl. No.: 13/893,666

(22) Filed: May 14, 2013

Related U.S. Application Data
(60) Provisional application No. 61/646,501, filed on May 14, 2012.

(51) Int. Cl. C07D 491/056

(52) U.S. Cl. CPC C07D 491/056
USPC 514/291

(57) ABSTRACT

A method of treating leukemia by administering an H4 agonist of histamine, 7-amino-4,5,6-triethoxy-3-(5,6,7,8-tetrahydro-4-methoxy-6-methyl-1,3-dioxolo[4,5-g]isoquinolin-5-yl) phthalide (tritoqualine), to a subject is provided.

### FIELD OF THE INVENTION

The invention relates the use of H4 agonist for the treatment of acute leukemia.

### BACKGROUND OF THE INVENTION

Blood cancers are diseases that can affect the bone marrow, the blood cells, the lymph nodes and other parts of the lymphatic system.

Leukemia is a malignant disease (cancer) of the bone marrow and blood. It is characterized by the uncontrolled accumulation of blood cells. Leukemia is divided into four categories: myelogenous or lymphocytic, each of which can be acute or chronic. The terms myelogenous or lymphocytic denote the cell type involved. There are four major types of leukemia:
Acute Myeloid Leukemia (AML)
Acute Lymphocytic Leukemia (ALL)
Chronic Myelogenous Leukemia (CML)
Chronic Lymphocytic Leukemia (CLL)

Expansion of myeloid blasts with suppression of normal hematopoiesis is a hallmark of acute myeloid leukemia (AML) but also acute lymphocytic leukemia.

In France the number of new acute leukemia is 4800 per year and 35 000 in Europe.

Overall incidence rates per 100,000 populations reported for leukemia are 12.3 in 2010 in US. There are 259,889 people living with, or in remission from leukemia in the US.

In 2010, 43,050 people will be diagnosed with leukemia and in 2010, 21,840 people will die of leukemia.

Leukemia causes about one-third of all cancer deaths in children younger than 15 years.

Several important new drugs have greatly improved rates of blood cancer cure and remission during the past decade. This new classes are:

### BCR-ABL Tyrosine Kinase Inhibitors

More than 90% cases Chronic Myeloid Leukemia is caused by chromosomal abnormality resulting in the formation of a so-called Philadelphia chromosome. This abnormality was discovered by Janet Rowley in 1972 and is due to fusion between Abelson tyrosine kinase gene at chromosome 9 and break point cluster (BCR) gene at chromosome 22, resulting in the chimeric oncongen Bcr-Abl and a constitutively active Bcr-Abl tyrosine kinase that has been implicated in the pathogenesis of CML. Compounds have been developed that selectively inhibit this tyrosine kinase.

Imatinib mesylate (Gleevec®), is the first generation drug since it is the first Bcr-Abl tyrosine kinase inhibitor to be used in the treatment. of CML. but imatinib have side effect such cough, diarrhea, dizziness, gas, hair loss, headache, increased tear production, loss of appetite, muscle cramps.

Due to increasing resistance and intolerance to imatinib efforts were made to develop new drugs that could inhibit the Bcr-Abl tyrosine kinase.

Nilotinib is a selective Bcr-Abl kinase inhibitor of second generation. Nilotinib is 10-30 fold more potent than imatinib in inhibiting activity of the Bcr-Abl tyrosine kinase and proliferation. But Nilotinib have a cardiac toxicity more important than Imatinib.

Dasatinib is also a selective Bcr-Abl kinase inhibitor of second generation and was tested in patents who were resistant to or who could not tolerate imatinib. But Dasatinib have many side effects such neutropenia, pleural effusion, diarrhea and hepatotoxicity.

But this class of drug has good result in chronic leukemia the result in acute leukemia are low,

### SUMMARY OF THE INVENTION

The invention provides a single diastereomeric structure comprised of two enantiomers, the RR and the SS of tritoqualine.

7-Amino-4,5,6-triethoxy-3-(5,6,7,8-tetrahydro-4-methoxy-6-methyl-1,3-dioxolo[4,5-g]isoquinolin-5-yl) phthalide or tritoqualine is a drug, currently formulated in 100 mg tablets and sold in pharmacies in Europe for the treatment of allergy.

Tritoqualine is not a pure product but. is available as a mixture of isomers.

The inventors have demonstrated that tritoqualine is a new histamine H4 agonist and, very surprisingly, with significant therapeutic activity on leukemia clone.

The invention also provides methods for treating acute leukemia.

### BRIEF DESCRIPTION OF THE FIGURES

FIG. 1 illustrates the chemical formula of tritoqualine (7-Amino-4,5,6-triethoxy-3-(5,6,7,8-tetrahydro-4-methoxy-6-methyl-1,3-dioxolo[4,5-g]isoquinolin-5-yl) phthalide)

FIG. 2 illustrates the sterical structure of the tritoqualine diastereomer D1.

FIG. 3 illustrates the sterical structure of the tritoqualine diastereoisomer D2

FIG. 4 illustrates the activity of tritoqualine against leukemia clones, TF1 clone.

FIG. 5 illustrates the activity of tritoqualine against HL60 leukemia clone.

FIG. 6 illustrates the activity of tritoqualine on OCT3, in comparison with another H4 histamine molecule (Clobenpropit)

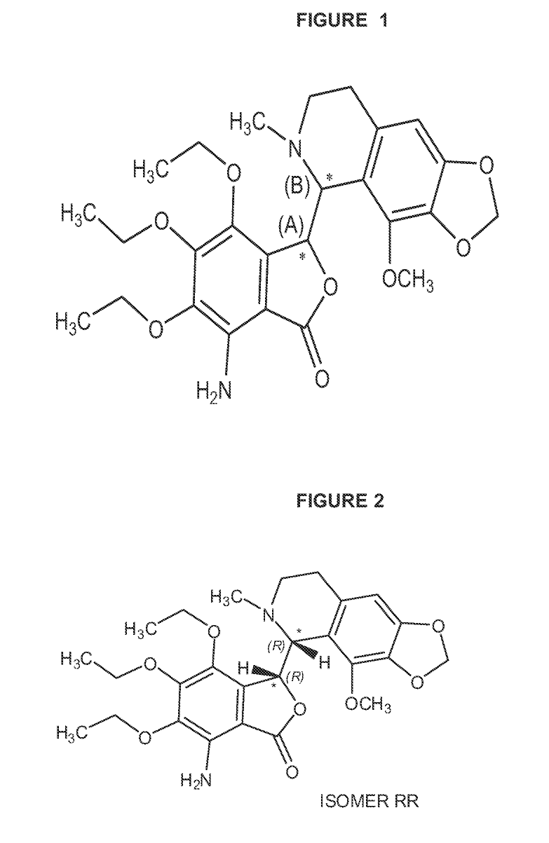

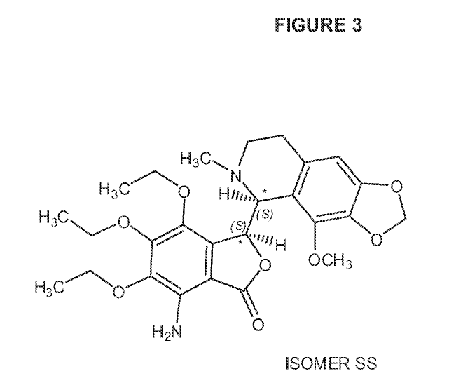

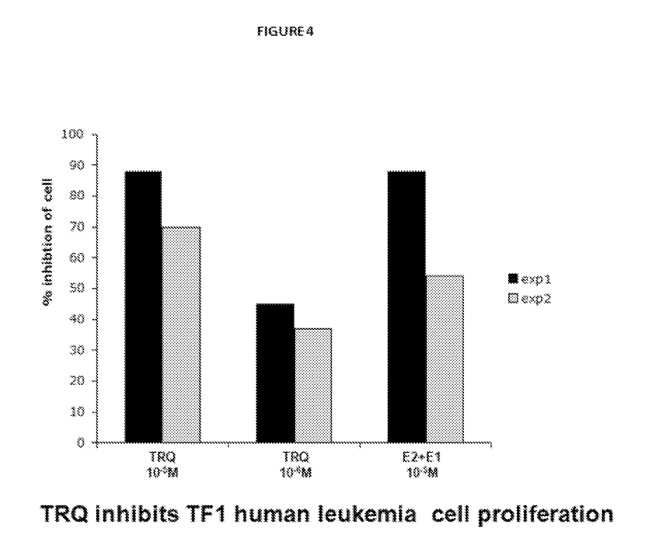

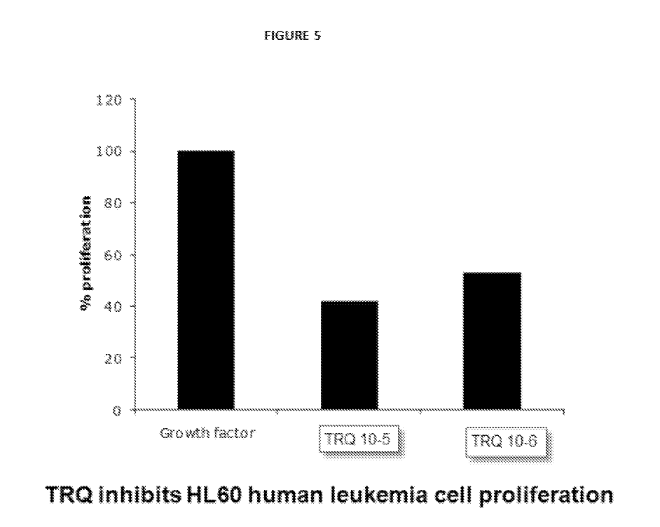

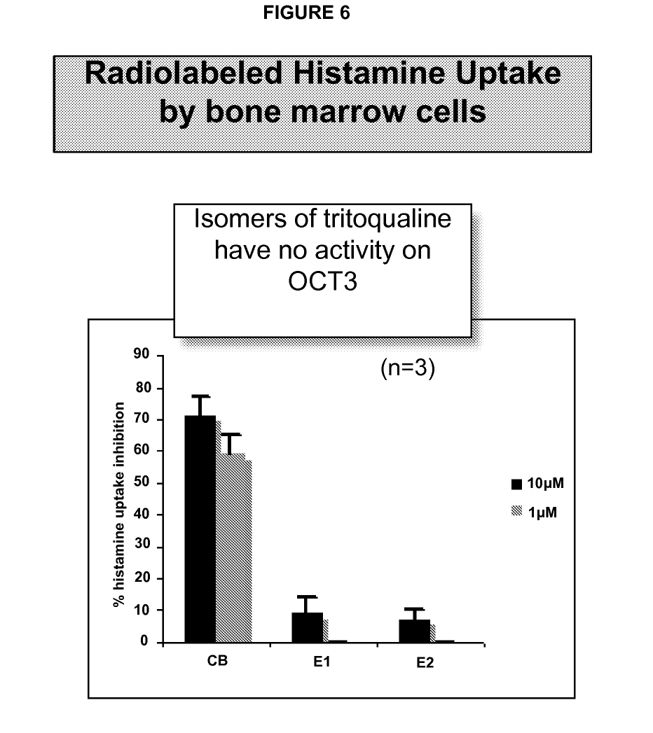

### DETAILED DESCRIPTION OF THE INVENTION

### Definitions

As used herein, the term “stereoisomers” referres to isomeric molecules whose atomic connectivity is the same but whose atomic arrangement in space is different.
As used herein, the term “enantiomers” referres to two chiral stereoisomers that are related to each other by a reflection. They are mirror images of each other and their atoms are nonsuperposable. Enantiomers have—when present in a symmetric environment—identical chemical and physical properties except for their ability to rotate plane-polarized light by equal amounts but in opposite directions. A solution of equal parts of an optically-active isomer and its enantiomer is known as a “racemic solution” or “racemate” and has a net rotation of plane-polarized light of zero.

### Cell Lines and Cell Culture

U937 (monocytic leukemia cells), and HL-60 (human promyelocytic cells) and TF1 (Human erythro leukemia cells) were obtained from cell bank of Necker.
HL-60, U937 and TF1 were maintained. in RPMI-1640 medium supplemented with 10% FCS, 100 U/mL penicillin, and 100 μg/mL streptomycin. These cells were cultured in an incubator containing humidified air with 5% CO2 at 3tC.

### Cell Proliferation Assays

HL60, U937, TF1 cells (5×105 cells/mL) in conditioning medium were cultured with various concentrations of tritoqualine (10⁻⁵-10⁻⁸ M) in the presence or absence of G-CSF (50 ng/mL) in 6-well flat-bottomed plates for 72 hr. Cell proliferation assay and viability examination were performed using the Trypan Blue dye exclusion method. All experiments were performed in triplicate.

### Cell-Cycle Analyses

To evaluate the cell cycle, we used an FITC BrdU Flow kit purchased from BD Pharmingen (San Jose, Calif.) according to the manufacturer's instructions. HL-60, and TF1 cells (5×105 cells/mL) were treated with tritoqualine (10-5 to 10-7 M) in the presence or absence of G-CSF (50 ng/mL) for 72 hrs.
Cultured cells were then labeled with BrdU for 45 min, washed, and fixed and permeabilized with BD Cytofix/Cytoperm Buffer.
After repeated incubation on ice (30 min, 10 min, and 5 min), washes, and centrifugation, cells were treated with DNase to expose BrdU epitope for 1 hour at 3tC, washed, then stained with fluorescent anti-BrdU for 20 min at room temperature, washed again, and centrifuged. Staining buffer containing 7-amino-actinomycin D (7-AAD) (1 mL; BD Pharmingen TM) was added to each tube to resuspend the cells, and the cells were analyzed by flow cytometry using FACScan (Becton Dickinson, San Jose, Calif.). Acquired multiparameter data were analyzed using CellQuest software.
With the combination of BrdU and 7-AAD, two-color flow cytometric analysis permits the enumeration and characterization of cells that are actively synthesizing DNA (BrdU incorporation) in terms of their cell cycle position.

### Proliferation Assay

We first examined the in vitro anti-leukemic effects of tritoqualine and confirmed that tritoqualine dose-dependently inhibited proliferation and viability of each leukemic cell line.
The maximum effect is on TF1 cells line. The inhibition is about 85%.
The effect on HL60 is only 60% of inhibition.
This effect dose is equivalent to the dose of 200 mg to 2000 mg of tritoqualine in human.
This effect on leukemia clone is very surprising because tritoqualine is a molecule with low toxicity.

1. A method of treating leukemia comprising administering an effective amount of a histamine H4 agonist to a subject in need thereof.

2. The method of claim 1, wherein the histamine H4 agonist is 7-amino-4,5,6-triethoxy-3-(5,6,7,8-tetrahydro-4-methoxy-6-methyl-1,3-dioxolo[4,5-g]isoquinolin-5-yl) phthalide.

3. The method of claim 1, wherein the 7-amino-4,5,6-triethoxy-3-(5,6,7,8-tetrahydro-4-methoxy-6-methyl-1,3-dioxolo[4,5-g]isoquinolin-5-yl) phthalide comprises the RR and SS isomers.

4. The method of claim 3, wherein the 7-amino-4,5,6-triethoxy-3-(5,6,7,8-tetrahydro-4-methoxy-6-methyl-1,3-dioxolo[4,5-g]isoquinolin-5-yl) phthalide is a racemic mixture.

5. The method of claim 1, wherein the 7-amino-4,5,6-triethoxy-3-(5,6,7,8-tetrahydro-4-methoxy-6-methyl-1,3-dioxolo[4,5-g]isoquinolin-5-yl) phthalide consists essentially of the RR isomer.

6. The method of claim 1, wherein the 7-amino-4,5,6-triethoxy-3-(5,6,7,8-tetrahydro-4-methoxy-6-methyl-1,3-dioxolo[4,5-g]isoquinolin-5-yl) phthalide consists essentially of the SS isomer.

7. The method of claim 1, wherein the leukemia is acute leukemia.

8. The method of claim 1, wherein the leukemia is erythroleukemia or myeloblastic leukemia without maturation.

9. The method of claim 1, wherein the H4 agonist is in the salt or hydrate form.

10. The method of claim 1, wherein the H4 agonist is administered to the subject in combination with a pharmaceutically acceptable carrier.

11. The method of claim 1, wherein the H4 agonist is administered to the subject at a dose of 300 to 2000 mg.

12. The method of claim 1, wherein the H4 agonist is administered to the subject in the form of a capsule, tablet, liquid or aerosol.

13. The method of claim 1, wherein the H4 agonist is administered to the subject orally, by injection or by inhalation.

14. The method of claim 1, wherein the subject is a human.
